(19) United States
(12) Patent Application Publication
(10) Pub. No.: US 2008/0188665 A1
(43) Pub. Date: Aug. 7, 2008

(54) Process for the preparation of 2-aminomethylpiperidine

(76) Inventors: Stefan Antons, Leverkusen (DE); Claus Dreisbach, Leichlingen (DE)

Correspondence Address: LANXESS CORPORATION, 111 RIDC PARK WEST DRIVE, PITTSBURGH, PA, 15275-1112, US

(21) Appl. No.: 12/005,008

(22) Filed: Dec. 20, 2007

(30) Foreign Application Priority Data
Dec. 27, 2006 (DE) 10 2006 061 533.6

(51) Int. Cl. C07D 211/26

(52) U.S. Cl. 546/185

(57) ABSTRACT

The invention relates to a process for the preparation of aminomethylpyridines of the general formula (I)

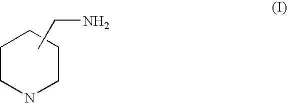

by hydrogenation of cyanopyridines of the general formula (II)

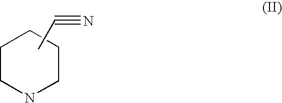

with hydrogen under increased pressure optionally in the presence of a catalyst containing Ni, Fe or Co and optionally in the presence of ammonia.

The invention relates to the preparation of 2-aminomethylpiperidine.

2-Aminomethylpiperidine is an essential building block for the production of pharmaceutical active ingredients. One of these pharmaceutical active ingredients is flecainide.

The production of flecainide is described, for example, in Chem. Ber.; GE; 118; 11; 1985; 4616-4619 and in J. Pharm. Sci.; EN; 80; 9; 1991; 887-890.

There has been no lack of attempts to prepare the 2-aminomethylpiperidine building block in the simplest and most economical manner possible. Thus, JACS (1941), p. 490 and JACS (1946), p. 1330 describe the preparation starting from 2-cyanopyridine. However, the described process is two-stage, the overall yield is only slightly above 20% of theory. Further serious disadvantages of the described procedure consist in the use of very expensive precious metal catalysts, such as, for example, Pt02. Also, the second reduction step is carried out in acetic acid or other mineral acids. Besides the possible risk of material corrosion, this has the major disadvantage that very much more additional effort is required to isolate the readily water-soluble 2-aminomethylpiperidine from its salts. The use of 2-aminomethylpyridine for the preparation of 2-aminomethylpiperidine is described in Tetrahedron Asymetry (1998, p. 1597); and in J. Pharm. Sci. (1990, pp. 750-53). However, here too, the problems and processing disadvantages described above are still present.

It was therefore the object to find a technically practicable and economical process which makes it possible, in a simple manner, to prepare 2-aminomethylpiperidine on an industrial scale.

Surprisingly, it has now been found that it is possible, starting from 2-cyanopyridine, to arrive at 2-aminomethylpiperidine in one step. It has also been found that the conversion is possible in a simple manner without the use of expensive precious metal catalysts and additional acids and also without the use of an additional solvent. The invention therefore provides a process for the preparation of aminomethylpyridines of the general formula (I)

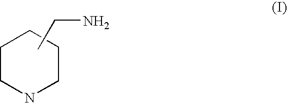

by hydrogenation of cyanopyridines of the general formula (II)

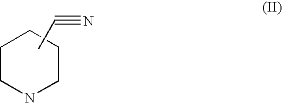

with hydrogen under increased pressure optionally in the presence of a catalyst containing Ni, Fe or Co and optionally in the presence of ammonia.

As a result of this, a very efficient and economic preparation of 2-aminomethylpiperidine becomes possible in which 2-cyanopyridine is hydrogenated directly without a diluent optionally in the presence of catalysts containing nickel and/or cobalt to give 2-aminomethylpiperidine. After filtering off the catalyst, the crude product is distilled such that pure 2-aminomethylpiperidine is obtained.

Preference is given to working at 100 to 200° C. and a pressure of from 50 to 300 bar.

### EXAMPLES

### Example 1 starting from 2-aminomethylpyridine

A mixture of 5 g of Ra—Ni 5584 and 100 g of 2-aminomethylpyridine was treated with 20 bar of hydrogen and then heated to 160° C. The hydrogen pressure was increased to 180 bar and hydrogenation was then carried out until hydrogen absorption is complete. After cooling and expansion, the system was aerated with nitrogen and the catalyst is filtered off. The resulting crude product had a 97% content of 2-aminomethylpiperidine.

### Example 2 starting from 2-aminomethylpyridine

A mixture of 10 g of Ra—Ni 5584 and 100 g of 2-aminomethylpyridine was treated with 20 bar of hydrogen and then heated to 200° C. The hydrogen pressure was increased to 160 bar and hydrogenation was then carried out until hydrogen absorption is complete. After cooling and expansion, the system was aerated with nitrogen and the catalyst was filtered off. The resulting crude product had a 94.5% content of 2-aminomethylpiperidine.

### Example 3 starting from 2-aminomethylpyridine

A mixture of 5 g of Ni—Fe-6606 and 100 g of 2-aminomethylpyridine was treated with 20 bar of hydrogen and then heated to 160° C. The hydrogen pressure was increased to 200 bar and hydrogenation was then carried out until hydrogen absorption is complete. After cooling and expansion, the system was aerated with nitrogen and the catalyst is filtered off. The resulting crude product had a 67.5% content of 2-aminomethylpiperidine.

### Example 4 starting from 2-cyanopyridine

A mixture of 25 g of Ni-5584 and 127.5 g of liquid ammonia was treated with 100 bar of hydrogen and heated to 180° C. After increasing the hydrogen pressure to 180 bar, 250 g of 2-cyanopyridine in 125 g of ethanol was added over the course of 6 hours. When the hydrogen absorption was complete, the system is cooled, expanded, and aerated with nitrogen, and the catalyst was filtered off. The resulting crude product had a 52% content of 2-aminomethylpiperidine.

1. Process for the preparation of aminomethylpyridines of the general formula (I) 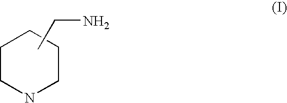
by hydrogenation of cyanopyridines of the general formula (II) 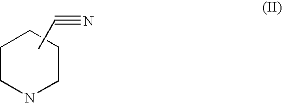
with hydrogen under increased pressure optionally in the presence of a catalyst containing Ni, Fe or Co and optionally in the presence of ammonia.

2. Process for the preparation of aminomethylpyridines according to claim 1, characterized in that the temperature is in the range from 10 to 200° C. and the pressure is in the range from 50 to 300 bar.

3. Process for the preparation of aminomethylpyridines according to claim 1, characterized in that catalysts containing Ni, Fe or Co are used.
